(19) United States
(12) Patent Application Publication
(10) Pub. No.: US 2012/0115944 A1
(43) Pub. Date: May 10, 2012

(54) USE OF CARNITINE AND DIHYDROQUERCITIN TO POSITIVELY INFLUENCE THE NATURAL PIGMENTATION PROCESS

(75) Inventors: Melanie Giesen, Geldern (DE); Edo Hoting, Hamburg (DE); Erik Schulze zur Wiesche, Hamburg (DE)

(73) Assignee: Henkel AG & Co., KGaA, Dusseldorf (DE)

(21) Appl. No.: 13/354,544

(22) Filed: Jan. 20, 2012

Related U.S. Application Data
(63) Continuation of application No. PCT/EP2010/060035, filed on Jul. 13, 2010.

(30) Foreign Application Priority Data
Jul. 23, 2009 (DE) 10 2009 027 957.1
Sep. 24, 2009 (DE) 10 2009 044 975.2

(51) Int. Cl. A61K 31/35; A61P 17/04; A61K 31/195

(52) U.S. Cl. 514/456; 514/561

(57) ABSTRACT

A method for positively influencing the natural pigmentation process of skin or appendages thereof includes topically contacting the skin or appendages thereof with a combination of carnitine and/or a derivative thereof with dihydroquercetin and/or a derivative thereof.

### CROSS-REFERENCES TO RELATED APPLICATIONS

This application is a continuation of PCT/EP2010/060035, filed on Jul. 13, 2010, which claims priority under 35 U.S.C. §119 to DE 10 2009 027 957.1 filed on Jul. 23, 2009, and DE 10 2009 044 975.2 filed on Sep. 24, 2009, all of which are hereby incorporated by reference.

### FIELD OF THE INVENTION

The present invention generally relates to products and methods for influencing the natural pigmentation process of skin and/or skin appendages.

### BACKGROUND OF THE INVENTION

Besides its actual physiological purpose, such as heat insulation and light protection, hair possesses a psychosocial function that must not be underestimated. Among other functions, it serves as a means of interpersonal communication, and represents a signal of one's own individuality. Changes such as, for example, graying can result in massive impairment of the self-image of the person involved.

The causes of graying hair have hitherto not been combated; instead, hair is treated, in order to cover gray, with the aid of chemical coloring agents that are often aggressive and thus damaging to the hair. In addition, customers often complain of poor compatibility (itching, burning, stinging) and durability (regular recoloring is necessary). The effectiveness of the few biological products presently on the market is not scientifically proven, and is often dubious. Significantly effective biologically active substances, which influence the graying process directly at the roots, are not in use.

Pigmentation in the hair follicle is controlled by a defined and complex set of molecular signals. Because melanogenesis is obviously influenced in grayed follicles, it may be assumed that the function of this network is modified in the grayed follicle. A consequent phenomenon is the decrease in melanin synthesis that results in graying of the follicle. Included among the complex set of molecular signals that influence melanogenesis are, among others, the expression of MCR1 (melanocortin receptor 1), gp100, and ckit. MCR1 and ckit are receptors that, as a result of the binding of their ligands (alpha-melanocyte stimulating hormone and stem cell factor), convey the key signals of melanogenesis into the interior of the cell. Gp100 is a protein of the melanosome membrane, and furthermore regulates additional proteins relevant to melanogenesis. Because these parameters are of essential significance in hair follicle pigmentation, it is advantageous to influence these parameters if the intention is to maintain or reactivate melanin synthesis in the hair follicle cells by application of test formulation. Maintaining the pigmentation, and thus youthfulness, of hair by means of suitable active-substance formulations is a challenge for cosmetic research.

It is known from the existing art to use dihydroquercetin in cosmetics because of its antioxidative properties. A negative effect on the natural pigmentation process of skin and/or skin appendages, resulting from an inhibition of the tyrosine activity necessary for melanin synthesis, is also discussed in the technical literature.

The patent EP 1845935 B1 claims the use of silybin, silymonin, silandrin, silychristin, silydianin, and isosylbin in dermatological compositions in order to induce, restore, or stimulate pigmentation of the skin, body hair, or head hair.

L-Carnitine is a vitamin-like active substance that is related to the B-complex vitamins and is essentially important for energy production and lipid metabolism in the human body. For this reason, L-carnitine is used chiefly as a nutritional supplement (US 2004/0170709 A1). Like L-carnitine, L-carnitine tartrate is also used as a nutritional supplement, for example to assist weight reduction (US 2004/0028668 A1).

The object of the present invention is to make available active substances that are suitable for influencing the natural pigmentation process, in particular in hair and/or hair follicles, without exhibiting the above-described disadvantages of the methods known in the existing art for positively influencing hair color and/or hair graying and the youthful appearance of hair.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

The above needs and others are met by a combination of carnitine and/or a carnitine derivative with dihydroquercetin and/or a dihydroquercetin derivative to positively influence the natural pigmentation process of skin and/or skin appendages.

More particularly, the above needs and others are met by a method for positively influencing the natural pigmentation process of skin and/or skin appendages, in particular for stimulating the natural pigmentation process, in particular the melanogenesis and/or pigmentation of hair, in order to prevent and/or decrease hair graying and/or to repigment grayed hair, wherein a combination of carnitine and/or a carnitine derivative with dihydroquercetin and/or a dihydroquercetin derivative is brought into contact topically with hair and/or skin. The above needs and others are also met by a hair treatment agent containing 0.1 to 90 wt % of at least one monovalent alcohol from the group of ethanol, n-propanol, isopropanol, n-butanol, 0 to 10 wt % of at least one gel former, L-carnitine and/or an L-carnitine derivative, and dihydroquercetin and/or a dihydroquercetin derivative.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Dihydroquercetin is a flavonoid (3,3′,4′,5,7-pentahydroxyflavanone) and is also known by the name “taxifolin.” Preferred dihydroquercetin derivatives exhibit the pentahydroxyflavanone basic skeleton and are etherified or esterified at one, two, or more hydroxy groups. Particularly preferred dihydroquercetin derivatives are dihydroquercetin monomethyl ether, dihydroquercetin dimethyl ether, and dihydroquercetin glycosides, in particular the glucosides, dihydroquercetin xylosides, dihydroquercetin rhamnosides, or dihydroquercetin galactosides. The O-3-glycosides, in which the hydroxy group at position 3 is glycosylated, are particularly preferred.

Dihydroquercetin and/or the dihydroquercetin derivative (hereinafter also referred to as dihydroquercetins) are preferably obtained as extracts. It is preferred in this context to use dihydroquercetin-containing extracts. Extracts of silymarin (milk thistle) that contain dihydroquercetin are used in particular.

The extracts of dihydroquercetins can be produced with water, as well as polar or nonpolar organic solvents, as well as mixtures thereof, in a manner known to one skilled in the art. Extracts that can be obtained by extraction with ethanol or water/ethanol mixtures, as well as compressed air, are preferred.

A first constituent of the combination to be used according to the present invention is carnitine (3-hydroxy-4-(trimethylammonium)butyric acid betaine, [(R-3-carboxy-2-hydroxypropyl]trimethylammonium betaine), particularly preferably L-carnitine.

Carnitine derivatives can furthermore also be used. Preferred carnitine derivatives are selected in particular from carnitine tartrate, acetylcarnitine, carnitine fumarate, carnitine citrate, lauroylcarnitine, and particularly preferably carnitine tartrate. The L-carnitine derivatives acetyl-L-carnitine, L-carnitine fumarate, L-carnitine citrate, lauroyl-L-carnitine, and particularly preferably L-carnitine tartrate are particularly preferred. The aforesaid L-carnitine compounds are obtainable, for example, from Lonza GmbH (Wuppertal, Germany).

According to a preferred embodiment, the ratio of the quantity of dihydroquercetin and/or dihydroquercetin derivative to the total quantity of L-carnitine and/or L-carnitine derivative is from 10:1 to 1:10, in particular from 7:1 to 1:7, preferably from 4:1 to 1:4.

Particularly preferably, the ratio of the quantity of dihydroquercetin to the total quantity of L-carnitine is from 10:1 to 1:10, in particular from 7:1 to 1:7, preferably from 4:1 to 1:4. Very particularly preferably, the ratio of the quantity of dihydroquercetin to the total quantity of L-carnitine tartrate is from 10:1 to 1:10, in particular from 7:1 to 1:7, preferably from 4:1 to 1:4.

According to a further preferred embodiment, dihydroquercetin and/or the dihydroquercetin derivative is used in a cosmetic agent that contains the dihydroquercetin and/or the dihydroquercetin derivative in a total quantity of 0.000001 to 3 wt %, preferably 0.00001 to 1 wt %, particularly preferably 0.0001 to 0.1 wt %, extraordinarily preferably 0.0003 to 0.05 wt %, based in each case on the total weight of the agent.

According to a further preferred embodiment, L-carnitine and/or the L-carnitine derivative is used in a cosmetic agent that contains carnitine and/or the L-carnitine derivative in a total quantity of 0.000001 to 10 wt %, preferably 0.00001 to 5 wt %, particularly preferably 0.0001 to 1 wt %, extraordinarily preferably 0.001 to 0.5 wt %, based in each case on the total weight of the agent.

Particularly preferred combinations are, in agents to be used according to the present invention, 0.000001 to 10 wt %, preferably 0.00001 to 5 wt %, particularly preferably 0.001 to 1 wt %, extraordinarily preferably 0.001 to 0.5 wt % L-carnitine, and 0.000001 to 3 wt %, preferably 0.00001 to 1 wt %, particularly preferably 0.0001 to 0.1 wt %, extraordinarily preferably 0.0003 to 0.05 wt % dihydroquercetin (taxifolin), based in each case on the total weight of the agent.

Very particularly preferred combinations are, in agents to be used according to the present invention, 0.000001 to 10 wt %, preferably 0.00001 to 5 wt %, particularly preferably 0.0001 to 1 wt %, extraordinarily preferably 0.001 to 0.5 wt % L-carnitine tartrate, and 0.000001 to 3 wt %, preferably 0.00001 to 1 wt %, particularly preferably 0.0001 to 0.1 wt %, extraordinarily preferably 0.0003 to 0.05 wt % dihydroquercetin (taxifolin), based in each case on the total weight of the agent.

It has been found, surprisingly, that the use of a combination of L-carnitine and/or an L-carnitine derivative with dihydroquercetin and/or the dihydroquercetin derivative is capable of positively influencing, in particular stimulating, the natural pigmentation process, in particular in hair and/or hair follicles. The combination according to the present invention induces both the gene expression of MCR-1 and that of ckit and gp100 in synergistic fashion. In addition, it has been possible to observe an enhancement of melanosynthesis. The natural pigmentation process is furthermore positively influenced by the increase in the available ATP concentration, and in that of hepatocyte growth factor (HGF) in the hair follicles.

The natural pigmentation process of skin and/or skin appendages can thus be influenced, in particular stimulated, by utilization of the combination and/or the agents incorporating the combination according to the present invention. In particular, the natural pigmentation process of hair, of the hair follicle, and/or in the hair follicle can thereby be influenced, in particular stimulated. The agents used according to the present invention are suitable for stimulating and/or improving the pigmentation of hair, stimulating melanogenesis (in particular in the hair follicle), preventing and/or decreasing hair graying, and repigmenting grayed hair.

The term “positively influencing the natural pigmentation process” is understood for purposes of the present invention to mean positively influencing the natural coloring/coloration and/or pigmentation of the skin and/or skin appendages, in particular stimulation of the natural, i.e. biological pigmentation process in the skin and/or skin appendages, in particular hair and/or hair follicles.

“Skin and skin appendages” are to be understood in connection with the present invention as the skin, mucous membranes, hair and its hair follicles, glands, and nails, in particular skin, mucous membranes, hair, and hair follicles. Particularly preferably, the term “skin” is to be understood as the skin without the mucous membranes. Very particularly preferably, the term “skin appendages” is to be understood as hair and/or hair follicles, preferably body hair, facial hair, and head hair, very particularly preferably facial hair and head hair, very particularly head hair, and/or the corresponding hair follicles.

According to a preferred embodiment, “positive influence on the natural pigmentation process” is understood to mean positive influence on at least one sub-step of the natural pigmentation process. This influence relates in particular to the regulation of those molecular signals that influence the biological and/or natural pigmentation process.

Regulation of the biological and/or natural pigmentation process by way of gene regulation, i.e. regulation at the expression level, and/or enzyme regulation, i.e. regulation at the activity level, and/or regulation at the hormone level, is preferred.

Regulation of melanogenesis, inter alia regulation of the gene expression of MCR1 (melanocortin receptor 1), gp100, and ckit, is particularly preferred. The regulation of tyrosinase, as well as gene expression of tyrosinase and regulation at the enzyme level, are also encompassed.

According to a preferred embodiment, the natural pigmentation process of the hair is influenced, in particular stimulated or excited. “Influence” is to be understood in particular as a positive influence, preferably positive regulation (upregulation and/or activation and/or excitation and/or elevation), which results in a stimulation of the natural, biological pigmentation process. Stimulation of melanogenesis in the human hair follicle, in particular of head hair (the hair follicles located on the scalp or top of the head) is particularly preferred.

The pigmentation process, in particular melanogenesis, of skin and skin appendages, preferably of hair and/or hair follicles can be influenced according to the present invention. In particular, the natural pigmentation process, in particular melanogenesis, can be influenced in mammals, particularly preferably in humans. Preferably, the pigmentation process, preferably melanogenesis, of human hair and/or of the human hair follicle is influenced.

“Stimulation of melanogenesis,” preferably of melanogenesis in the hair follicle, is to be understood particularly preferably according to the present invention as the stimulation, elevation, excitation, and/or improvement of melanin synthesis in the melanocytes (preferably the melanocytes in the hair follicle). This is achieved, for example, by an elevation of the gene expression of signal molecules such as MCR1 (melanocortin receptor 1), gp100, and ckit. According to a preferred embodiment, the positive influence, preferably stimulation, of melanogenesis is achieved by way of the use according to the present invention. Melanogenesis is stimulated in particular in the hair and/or hair follicle of the hair-covered scalp and/or of the beard, in particular in humans.

For purposes of the present invention, “stimulation of pigmentation” is to be understood in particular as improvement, elevation, and/or stimulation of the transport of melanosomes into the keratinocytes surrounding the hair follicle, and furthermore as the pigmentation, perceptible with the eye or with correspondingly suitable measurement methods, of the individual hair, of a selection of hairs, in particular an area of hair-covered skin, in particular scalp, or of the entirety of the head hair and/or facial hair.

In the context of a preferred embodiment, hair graying, in particular of human hair, is prevented, preferably substantially prevented, and/or decreased by the use according to the present invention. “Hair graying” is to be understood for purposes of the present invention both as visually perceptible hair graying as a result of the mixing of white and pigmented hairs, and as pigment dilution in a single hair, i.e. the graying of a single hair.

A prevention of hair graying occurs in particular in hair that is not yet grayed; a decrease in hair graying can take place both in hair that is already grayed and in hair that is not yet grayed. In the one case, melanogenesis is excited/stimulated again in hair follicles in which melanogenesis is not, is no longer, or is not completely functioning and/or is disrupted or reduced, while in the non-grayed hair/hair follicles, a disruption, reduction, and/or downregulation of melanogenesis takes place not at all or only to a lesser degree.

According to a further preferred embodiment, already-grayed hair is repigmented by means of the use according to the present invention of a combination of L-carnitine and/or an L-carnitine derivative with dihydroquercetin and/or a dihydroquercetin derivative.

According to a further particularly preferred embodiment, the use according to the present invention is a cosmetic use that is non-therapeutic.

In particular, the use according to the present invention which is directed toward hair graying that results from the natural aging process, in particular that is not disease-related, is a purely cosmetic use that does not represent treatment and/or prophylaxis of a disease and is thus non-therapeutic.

According to a particular embodiment, the use according to the present invention occurs topically, i.e. by application onto the skin and/or skin appendage, in particular the facial and/or head hair, in particular head hair.

The cosmetic agents according to the present invention exhibit improved care effects on the skin and hair. The positive effects are quite pronounced particularly on keratinic fibers, so that preferred cosmetic agents according to the present invention are hair treatment agents.

Hair treatment agents for purposes of the present invention are, for example, hair coloring agents, hair bleaching agents, hair shampoos, hair conditioners, conditioning shampoos, hair sprays, hair rinses, hair therapies, hair packs, hair tonics, permanent wave fixing solutions, hair coloring shampoos, hair coloring agents, hair setting agents, hairstyling agents, hairstyling preparations, blow-dry lotions, foam setting agents, hair gels, hair waxes, or combinations thereof. Particularly preferred hair treatment agents are characterized in that they are packaged as a shampoo, hair tonics, hair therapy, hair rinse, hair foam, hair setting agent, hair spray, hair gel, and/or hair coloring agent. These agents are particularly advantageous in view of the fact that for reasons of time and convenience, the consumer is often reluctant to use several different agents and/or multiple application steps.

According to a preferred embodiment, at least one hair conditioning agent, selected from cationic polymers, cationic surfactants, silicones, and/or vegetable oils, is additionally preferably contained.

The agents used according to the present invention can contain further active substances and adjuvants. These are described below.

The compositions to be used according to the present invention can contain surfactants, in particular cationic surfactants. Protection is sought, or protection may be sought, for surfactant-containing agents to be used according to the present invention; surfactants, in particular cationic surfactants, contribute to the technical objective of the invention and thus to achieving the technical object on which the invention according to the Application is based. Preferred surfactants, the quantities in which they are contained in compositions according to the present invention, are disclosed in the priority document DE 10 2009 044975 on pages 7 to 18; the features recited therein unequivocally belong implicitly to the description of the invention contained in the Application submitted, and thus to the disclosure of this Application. Particularly preferred hair treatment agents according to the present invention are characterized in that they contain as a cationic care-providing substance, based on their weight, 0.05 to 7.5 wt %, by preference 0.1 to 5 wt %, particularly preferably 0.2 to 3.5 wt %, and in particular 0.25 to 2.5 wt % cationic surfactant(s) from the group of the quaternary ammonium compounds and/or the esterquats and/or the amidoamines, preferred cationic surfactant(s) being selected from alkyltrimethylammonium chlorides having 10 to 18 carbon atoms in the alkyl residue and/or dialkyldimethylammonium chlorides having by preference 10 to 18 carbon atoms in the alkyl residue and/or trialkylmethylammonium chlorides having by preference 10 to 18 carbon atoms in the alkyl residue and/or cetyltrimethylammonium chloride and/or stearyltrimethylammonium chloride and/or distearyldimethylammonium chloride and/or lauryldimethylammonium chloride and/or lauryldimethylbenzylammonium chloride and/or tricetylmethylammonium chloride and/or Quaternium-27 and/or Quaternium-83 and/or N-methyl-N(2-hydroxyethyl)-N,N-(ditallowacyloxyethyl)ammonium methosulfate and/or N-methyl-N(2-hydroxyethyl)-N,N-(distearoyloxyethyl)ammonium methosulfate and/or N,N-dimethyl-N,N-distearoyloxyethylammonium chloride and/or N,N-di-(2-hydroxyethyl)-N,N-(fatty acid ester ethyl)ammonium chloride.

As a further optional constituent, the agents used according to the present invention can contain 0.01 to 10 wt % of at least one polymer from the group of the cationic and/or amphoteric polymers. Protection is sought, or protection may be sought, for polymer-containing agents to be used according to the present invention; polymers, in particular cationic polymers, contribute to the technical objective of the invention and thus to achieving the technical object on which the invention according to the Application is based. Preferred polymers, the quantities in which they are contained in compositions to be used according to the present invention, are disclosed in the priority document DE 10 2009 044975 on pages 18 to 27; the features recited therein unequivocally belong implicitly to the description of the invention contained in the Application submitted, and thus to the disclosure of this Application.

A further preferred group of ingredients of the agents used according to the present invention are the vitamins, provitamins, or vitamin precursors. These are described below:

The group of substances referred to as vitamin A includes retinol (vitamin A₁) as well as 3,4-didehydroretinol (vitamin A₂). ®-Carotene is the provitamin of retinol. Vitamin A components that are suitable according to the present invention are, for example, vitamin A acid and esters thereof, vitamin A aldehyde, and vitamin A alcohol as well as esters thereof such as the palmitate and acetate. The agents used according to the present invention contain the vitamin A component preferably in quantities from 0.05 to 1 wt % based on the entire preparation.

Members of the vitamin B group or vitamin B complex are, among others, vitamin B₁ (thiamine), vitamin B₂ (riboflavin), vitamin B₃. The compounds nicotinic acid and nicotinic acid amide (niacinamide) are often listed under this designation. Nicotinic acid amide is preferred according to the present invention; it is contained in the agents used according to the present invention preferably in quantities from 0.05 to 1 wt % based on the entire agent. Also included thereamong is vitamin B₅ (pantothenic acid, panthenol, and pantolactone). Panthenol and/or pantolactone are preferably used in the context of this group. Derivatives of panthenol usable according to the present invention are, in particular, the esters and ethers of panthenol as well as cationically derivatized panthenols. Individual representatives are, for example, panthenol triacetate, panthenol monoethyl ether and the monoacetate thereof, as well as the cationic panthenol derivatives disclosed in WO 92/13829. The aforesaid compounds of the vitamin B₅ type are contained in the agents used according to the present invention preferably in quantities from 0.05 to 10 wt % based on the entire agent. Quantities from 0.1 to 5 wt % are particularly preferred. Vitamin B₆ (pyridoxine as well as pyridoxamine and pyridoxal) can furthermore be used. Vitamin C (ascorbic acid). Vitamin C is utilized in the agents used according to the present invention preferably in quantities from 0.1 to 3 wt % based on the entire agent. Utilization in the form of the palmitic acid ester, the glucosides or phosphates can be preferred. Utilization in combination with tocopherols can likewise be preferred. Vitamin E (tocopherols, in particular (-tocopherol). Tocopherol and derivatives thereof, which include in particular the esters such as the acetate, nicotinate, phosphate, and succinate, are contained in the agents used according to the present invention preferably in quantities from 0.05 to 1 wt % based on the agent. Vitamin F. The term “vitamin F” is usually understood as essential fatty acids, in particular linoleic acid, linolenic acid, and arachidonic acid. Vitamin H. “Vitamin H” refers to (3aS,4S,6aR)-2-oxohexahydrothienol[3,4-d]-imidazole-4-valeric acid, for which the trivial name “biotin” has nevertheless since become established. Biotin is contained in the agents used according to the present invention preferably in quantities from 0.0001 to 1.0 wt %, in particular in quantities from 0.001 to 0.01 wt %.

Combinations of dihydroquercetin and L-carnitine with tocopherols, in particular alpha-tocopherol, as well as dihydroquercetin and L-carnitine tartrate with tocopherols, in particular alpha-tocopherol, are particularly preferred.

Particularly preferred hair treatment agents used according to the present invention are characterized in that they contain as a care-providing substance, based on their weight, 0.0001 to 1 wt %, preferably 0.001 to 0.5 wt %, and particularly preferably 0.005 to 0.1 wt % of at least one ubiquinone and/or at least one ubiquinol and/or at least one derivative of said substances; agents to be used with particular preference contain coenzyme Q10, preferably at 0.005 to 1.1 wt %. Alternatively to the particularly preferred ubiquinones or in addition to them, the agents used according to the present invention can also contain plastoquinones (polyprenylated 2,3-dimethylbenzoquinone derivatives). Preferred agents used according to the present invention are characterized here in that they contain 0.0002 to 4 wt %, by preference 0.0005 to 3 wt %, particularly preferably 0.001 to 2 wt %, more preferably 0.0015 to 1, and in particular 0.002 to 0.5 wt % of at least one plastoquinone. The prenyl side chain contains, in this context, n prenyl units. Values for n are preferably from 1 to 20, by preference from 2 to 15, and in particular denotes 5, 6, 7, 8, 9, 10, agents particularly preferred for use containing a plastoquinone where n=9. A combination of dihydroquercetin, L-carnitine, and coenzyme Q10 is particularly preferred; a combination of dihydroquercetin, L-carnitine tartrate, and coenzyme Q10 is further preferred.

As a further ingredient, the agents used according to the present invention can, with particular preference, contain one or more amino acids. Amino acids usable particularly preferably according to the present invention derive from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cys), L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, in which context both the individual amino acids and mixtures can be used. Preferred agents used according to the present invention contain one or more amino acids in narrower quantity ranges. Hair treatment agents preferred according to the present invention are here characterized in that they contain as a care-providing substance, based on their weight, 0.01 to 5 wt %, by preference 0.02 to 2.5 wt %, particularly preferably 0.05 to 1.5 wt %, more preferably 0.075 to 1 wt %, and in particular 0.1 to 0.25 wt % amino acid(s), by preference from the group of glycine and/or alanine and/or valine and/or lysine and/or leucine and/or threonine.

A combination of dihydroquercetin and L-carnitine with glycine, dihydroquercetin and L-carnitine with alanine, dihydroquercetin and L-carnitine with valine, dihydroquercetin and L-carnitine with lysine, dihydroquercetin and L-carnitine with leucine, dihydroquercetin and L-carnitine with threonine is particularly preferred. Also particularly preferred are combinations of dihydroquercetin and L-carnitine tartrate with glycine, dihydroquercetin and L-carnitine tartrate with alanine, dihydroquercetin and L-carnitine tartrate with valine, dihydroquercetin and L-carnitine tartrate with lysine, dihydroquercetin and L-carnitine tartrate with leucine, dihydroquercetin and L-carnitine tartrate with threonine.

Agents to be used preferably according to the present invention contain as a care-providing substance, based on their weight, 0.01 to 15 wt %, by preference 0.025 to 12.5 wt %, particularly preferably 0.05 to 10 wt %, more preferably 0.1 to 7.5 wt %, and in particular 0.5 to 5 wt % of at least one 2-furanone derivative of formula (Fur-I) and/or formula (Fur-II)

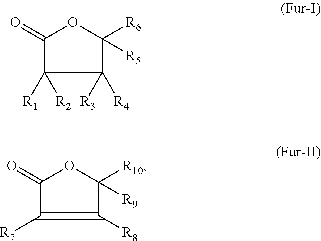

Protection is sought, or protection may be sought, for furanone-derivative-containing agents to be used according to the present invention; 2-furanone derivatives contribute to the technical objective of the invention and thus to achieving the technical object on which the invention according to the Application is based. Suitable 2-furanone derivatives, the quantities in which they are contained in compositions to be used according to the present invention, are disclosed in the priority document DE 10 2009 044975 on pages 33 to 38; the features recited therein unequivocally belong implicitly to the description of the invention contained in the Application submitted, and thus to the disclosure of this Application.

A further, preferably usable care-providing substance that possesses activating properties is taurine. Hair treatment agents preferred according to the present invention contain as a care-providing substance, based on their weight, 0.01 to 15 wt %, by preference 0.025 to 12.5 wt %, particularly preferably 0.05 to 10 wt %, more preferably 0.1 to 7.5 wt %, and in particular 0.5 to 5 wt % taurine (2-aminoethanesulfonic acid).

The agents used according to the present invention can contain, in addition to optional further ingredients, further substances that prevent, mitigate, or cure hair loss. A content of hair-root-stabilizing active substances is particularly advantageous. These substances are described below: Propecia (finasteride) is at present the only preparation that is approved worldwide and for which effectiveness and compatibility have been demonstrated in numerous studies. The effect of Propecia is that less DHT can form from testosterone. Minoxidil, with or without supplementary additives, is probably the oldest demonstrably effective hair growth agent. For the treatment of hair loss, it may be used only for external application. Hair lotions exist that contain 2% to 5% minoxidil, also gels having up to 15% minoxidil. Effectiveness increases with dosage, but minoxidil is soluble in hair lotions only up to a 5% proportion. In many countries, hair lotions with a minoxidil content of up to 2% are obtainable without a prescription. To counteract hormonal influences on the hair follicles, spironolactone, in the form of hair lotion and in combination with minoxidil, can be applied for external use. Spironolactone acts as an androgen receptor blocker, i.e. the binding of DHT to the hair follicles is prevented. Cosmetic agents to be used according to the present invention that additionally contain, based on its weight, 0.001 to 5 wt % hair-root-stabilizing substances, in particular minoxidil and/or finasteride and/or ketoconazole, are particularly preferred. The agents used according to the present invention can further contain all active substances, additives, and adjuvants known for such preparations. In many cases the agents contain at least one surfactant; in principle, both anionic as well as zwitterionic, ampholytic, nonionic, and cationic surfactants are suitable. In many cases, however, it has proven advantageous to select the surfactants from anionic, zwitterionic, or nonionic surfactants. These surfactants have already been described in detail above.

A preferred presentation form of the hair treatment agent according to the present invention is in the form of hair tonics or hair lotions. These preferably contain at least one monovalent alcohol, L-carnitine and/or an L-carnitine derivative, as well as dihydroquercetin and/or a dihydroquercetin derivative, optionally a gel former, and optionally at least one specific care enhancer.

In a further embodiment, a subject of the present invention is a hair treatment agent containing

(a) 0.1 to 90 wt % of at least one monovalent alcohol from the group of ethanol, n-propanol, isopropanol, n-butanol,
(b) 0 to 10 wt % of at least one gel former,
(c) L-carnitine and/or an L-carnitine derivative, and
(d) dihydroquercetin and/or a dihydroquercetin derivative.

With regard to further preferred embodiments of the agent according to the present invention, the statements made about the uses according to the present invention apply mutatis mutandis.

A hair treatment agent containing

(a) 0.1 to 90 wt % of at least one monovalent alcohol from the group of ethanol, n-propanol, isopropanol, n-butanol,
(b) 0 to 10 wt % of at least one gel former,
(c) L-carnitine tartrate, and
(d) dihydroquercetin
is particularly preferred.

The agents used according to the present invention contain 0.1 to 90 wt % of at least one monovalent alcohol from the group of ethanol, n-propanol, isopropanol, n-butanol. Among these, ethanol and/or isopropanol are particularly preferred. Particularly preferred hair treatment agents according to the present invention are characterized in that they contain, based on their weight, 0.5 to 85 wt %, by preference 1 to 80 wt %, particularly preferably 5 to 75 wt %, more preferably 10 to 70 wt %, and in particular 25 to 60 wt % ethanol and/or isopropanol.

Particularly preferred hair treatment agents contain exclusively ethanol. Hair treatment agents that contain, based on their weight, 5 to 80 wt %, by preference 7.5 to 70 wt %, particularly preferably 10 to 60 wt %, more preferably 20 to 55 wt %, and in particular 25 to 50 wt % ethanol, are particularly preferred here.

The agents used according to the present invention can additionally contain a gel former. As a result of the use of these gel formers, adhesion of the agents onto the hair can be improved, and application can be made more pleasant. Hair treatment agents according to the present invention that contain, based on their weight, 0.15 to 9 wt %, by preference 0.2 to 8 wt %, particularly preferably 0.25 to 7 wt %, more preferably 0.3 to 6 wt %, and in particular 0.4 to 5 wt % of at least one gel former from the groups of the silicic acids and/or sheet silicates and/or sheet organosilicates, and/or metal soaps and/or hardened castor oil and/or modified fat derivatives and/or polyamides and/or hydroxyethyl cellulose (HEC) and/or carboxymethyl cellulose (CMC) and/or hydroxypropylmethyl cellulose (HPMC) and/or hydroxypropyl cellulose (HPC) and/or ethylhydroxyethyl cellulose (EHEC) and/or polyvinyl alcohols and/or polyacrylic acid and/or polymethacrylic acids and salts thereof, and/or polyacrylamides and/or polyvinylpyrrolidone and/or polyethylene glycols and/or styrene-maleic acid anhydride copolymerizates and salts thereof, and/or copolymers and/or terpolymers of acrylic acid and methacrylic acid and/or cellulose and/or starch and/or xanthan, are preferred here.

In a further preferred embodiment, the agents used according to the present invention, in particular including the hair lotions and/or hair tonics according to the present invention, can contain emulsifiers (F). Protection is sought, or protection may be sought, for emulsifier-containing agents to be used according to the present invention; emulsifiers contribute to the technical objective of the invention and thus to achieving the technical object on which the invention according to the Application is based. Preferred emulsifiers, the quantities in which they are contained in compositions according to the present invention, are disclosed in the priority document DE 10 2009 044975 on pages 41 to 42; the features recited therein unequivocally belong implicitly to the description of the invention contained in the Application submitted, and thus to the disclosure of this Application.

In a preferred embodiment of the invention, an agent according to the present invention can furthermore also contain UV filters (I). The UV filters to be used according to the present invention are not subject to any general restrictions in terms of their structure and their physical properties. Instead, all UV filters usable in the cosmetics sector, whose absorption maximum lies in the UVA (315 to 400 nm) UVB (280 to 315 nm), or UVC (<280 nm) regions, are suitable. UV filters having an absorption maximum in the UVB region, in particular in the region from approximately 280 to approximately 300 nm, are particularly preferred. The UV filters used according to the present invention can be selected, for example, from substituted benzophenones, p-aminobenzoic acid esters, diphenylacrylic acid esters, cinnamic acid esters, salicylic acid esters, benzimidazoles, and o-aminobenzoic acid esters. Examples of UV filters usable according to the present invention are 4-aminobenzoic acid, N,N,N-trimethyl-4-(2-oxoborn-3-ylidenemethyl)aniline methylsulfate, 3,3,5-trimethylcyclohexyl salicylate (Homosalate), 2-hydroxy-4-methoxybenzophenone (Benzophenone-3; Uvinul® M 40, Uvasorb® MET, Neo Heliopan® BB, Eusolex® 4360), 2-phenylbenzimidazole-5-sulfonic acid and potassium, sodium, and triethanolamine salts thereof (phenylbenzimidazolesulfonic acid; Parsol® HS; Neo Heliopan® Hydro), 3,3′-(1,4-phenylenedimethylene)-bis(7,7-dimethyl-2-oxo-bicyclo-[2.2.1]hept-1-yl-methanesulfonic acid) and salts thereof, 1-(4-tert-butylphenyl)-3-(4-methoxyphenyl)propane-1,3-dione (butylmethoxydibenzoylmethane; Parsol® 1789, Eusolex® 9020), α-(2-oxoborn-3-ylidene)toluene-4-sulfonic acid and salts thereof, ethoxylated 4-aminobenzoic acid ethyl ester (PEG-25 PABA; Uvinul® P 25), 4-dimethylaminobenzoic acid 2-ethylhexyl ester (Octyl Dimethyl PABA; Uvasorb® DMO, Escalol® 507, Eusolex® 6007), salicylic acid 2-ethylhexyl ester (Octyl Salicylate; Escalol® 587, Neo Heliopan® OS, Uvinul® O18), 4-methoxycinnamic acid isopentyl ester (Isoamyl p-Methoxycinnamate; Neo Heliopan® E 1000), 4-methoxycinnamic acid 2-ethylhexyl ester (Octyl Methoxycinnamate; Parsol® MCX, Escalol® 557, Neo Heliopan® AV), 2-hydroxy-4-methoxybenzophenone-5-sulfonic acid and the sodium salt thereof (Benzophenone-4; Uvinul® MS 40; Uvasorb® S 5), 3-(4′-methylbenzylidene) D,L-camphor (4-Methylbenzylidene Camphor; Parsol® 5000, Eusolex® 6300), 3-benzylidene camphor (3-Benzylidene Camphor), 4-isopropylbenzyl salicylate, 2,4,6-trianilino-(p-carbo-2′-ethylhexyl-1′-oxi)-1,3,5-triazine, 3-imidazol-4-ylacrylic acid and ethyl esters thereof, polymers of N-{(2 and 4)-[2-oxoborn-3-ylidenemethyl]benzyl}acrylamide, 2,4-dihydroxybenzophenone (Benzophenone-1; Uvasorb® 20 H, Uvinul® 400), 1,1′-diphenylacrylonitrilic acid 2-ethylhexyl ester (Octocrylene; Eusolex® OCR, Neo Heliopan® Type 303, Uvinul® N 539 SG), o-aminobenzoic acid menthyl ester (Menthyl Anthranilate; Neo Heliopan® MA), 2,2′,4,4′-tetrahydroxybenzophenone (Benzophenone-2; Uvinul® D-50), 2,2′-dihydroxy-4,4′-dimethoxybenzophenone (Benzophenone-6), 2,2′-dihydroxy-4,4′-dimethoxybenzophenone-5-sodiumsulfonate, and 2-cyano-3,3-diphenylacrylic acid 2′-ethylhexyl ester. 4-Aminobenzoic acid, N,N,N-trimethyl-4-(2-oxoborn-3-ylidenemethyl)aniline methyl sulfate, 3,3,5-trimethylcyclohexyl salicylate, 2-hydroxy-4-methoxybenzophenone, 2-phenylbenzimidazole-5-sulfonic acid and potassium, sodium, and triethanolamine salts thereof, 3,3′-(1,4-phenylenedimethylene)-bis(7,7-dimethyl-2-oxo-bicyclo-[2.2.1]hept-1-ylmethanesulfonic acid) and salts thereof, 1-(4-tert.-butylphenyl)-3-(4-methoxyphenyl)propane-1,3-dione, α-(2-oxoborn-3-ylidene)toluene-4-sulfonic acid and salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone-5-sulfonic acid and the sodium salt thereof, 3-(4′-methylbenzylidene) D,L-camphor, 3-benzylidene camphor, 4-isopropylbenzyl salicylate, 2,4,6-trianilino-(p-carbo-2′-ethylhexyl-1′-oxi)-1,3,5-triazine, 3-imidazol-4-ylacrylic acid and ethyl esters thereof, and polymers of N-{(2 and 4)-[2-oxoborn-3-ylidenemethyl]benzyl}acrylamide are preferred. Very particularly preferred according to the present invention are 2-hydroxy-4-methoxybenzophenone, 2-phenylbenzimidazole-5-sulfonic acid and potassium, sodium, and triethanolamine salts thereof, 1-(4-tert.-butylphenyl)-3-(4-methoxyphenyl)propane-1,3-dione, 4-methoxycinnamic acid 2-ethylhexyl ester, and 3-(4′-methylbenzylidene) D,L-camphor. Those UV filters whose molar extinction coefficient at the absorption maximum is above 15,000, in particular above 20,000, are preferred. It has furthermore been found that with structurally similar UV filters, in the context of the teaching of the present invention the water-insoluble compound in many cases exhibits the greater effectiveness as compared with those water-soluble compounds that differ from it by having one or more additionally ionic groups. In the context of the invention, those UV filters of which no more than 1 wt %, in particular no more than 0.1 wt %, dissolves in water at 20° C., are understood to be “water-insoluble.” These compounds should furthermore be soluble at a proportion of at least 0.1, in particular at least 1 wt %, in usual cosmetic oil components at room temperature. The use of water-insoluble UV filters can therefore be preferred according to the present invention. According to a further embodiment of the present invention, those UV filters that comprise a cationic group, in particular a quaternary ammonium group, are preferred. These UV filters exhibit the general structure U-Q.

The structural part U denotes a group that absorbs UV radiation. This group can be derived in principle from the aforementioned known UV filters usable in the cosmetics sector, in which one group, generally a hydrogen atom, of the UV filter is replaced by a cationic group Q, in particular by a quaternary amino function.

Compounds from which the structural part U can be derived are, for example, substituted benzophenones, p-aminobenzoic acid esters, diphenylacrylic acid esters, cinnamic acid esters, salicylic acid esters, benzimidazoles, and o-aminobenzoic acid esters.

Structural parts U that are derived from cinnamic acid amide or from N,N-dimethylaminobenzoic acid amide are preferred according to the present invention.

The structural parts U can in principle be selected so that the absorption maximum of the UV filters can lie both in the UVA (315 to 400 nm) region and in the UVB (280 to 315 nm) region, or in the UVC (<280 nm) region. UV filters having an absorption maximum in the UVB region, in particular in the region from approximately 280 to approximately 300 nm, are particularly preferred.

The structural part U is furthermore preferably selected, including as a function of the structural part Q, in such a way that the molar extinction coefficient of the UV filter at the absorption maximum is above 15,000, in particular above 20,000.

The structural part Q preferably contains a quaternary ammonium group as a cationic group. This quaternary ammonium group can in principle be connected directly to the structural part U, so that the structural part U represents one of the four substituents of the positively charged nitrogen atom. Preferably, however, one of the four substituents on the positively charged nitrogen atom is a group, in particular an alkylene group, having 2 to 6 carbon atoms, which functions as a connection between the structural part U and the positively charged nitrogen atom.

Advantageously, the group Q has the general structure —(CH₂)_{X}—N⁺R¹R²R³X⁻, in which x denotes an integer from 1 to 4, R¹ and R², mutually independently, denote C₁₋₄ alkyl groups, R³ denotes a C₁₋₂₂ alkyl group or a benzyl group, and X⁻ denotes a physiologically acceptable anion. In the context of this general structure, x preferably denotes the number 3, R¹ and R² each denote a methyl group, and R³ denotes either a methyl group or a saturated or unsaturated, linear or branched hydrocarbon chain having 8 to 22, in particular 10 to 18, carbon atoms.

Physiologically acceptable anions are, for example, inorganic anions such as halides, in particular chloride, bromide and fluoride, sulfate ions, and phosphate ions, as well as organic anions such as lactate, citrate, acetate, tartrate, methosulfate, and tosylate.

Two preferred UV filters having cationic groups are the compounds cinnamic acid amidopropyltrimethylammonium chloride (Incroquat® UV-283) and dodecyldimethylaminobenzamidopropyldimethylammonium tosylate (Escalol® HP 610), available as commercial products.

The teaching of the present invention of course also encompasses the use of a combination of several UV filters. In the context of this embodiment, the combination of at least one water-insoluble UV filter with at least one UV filter having a cationic group is preferred. The UV filters are contained in the agents used according to the present invention usually in quantities from 0.1 to 5 wt % based on the entire agent. Quantities from 0.4 to 2.5 wt % are preferred. The UV filters in the agents used according to the present invention improve the results of the repigmentation process, in particular over the long term, and are therefore particularly suitable. Particularly preferably, at least one of the UV filters recited above is combined with dihydroquercetin and L-carnitine or L-carnitine tartrate.

It has proven to be further advantageous if, in addition to the polymer(s) from the group of the cationic and/or amphoteric polymers, further polymers (G) are contained in the agents used according to the present invention. Protection is sought, or protection may be sought, for polymer-containing agents according to the present invention; polymers contribute to the technical objective of the invention and thus to achieving the technical object on which the invention according to the Application is based. Preferred polymers, the quantities in which they are contained in compositions used according to the present invention, are disclosed in the priority document DE 10 2009 044975 on pages 42 to 44; the features recited therein unequivocally belong implicitly to the description of the invention contained in the Application submitted, and thus to the disclosure of this Application.

The agents used according to the present invention can furthermore contain a 2-pyrrolidinone-5-carboxylic acid and derivatives thereof (J). The sodium, potassium, calcium, magnesium, or ammonium salts, in which the ammonium ion carries, besides hydrogen, one to three C₁ to C₄ alkyl groups, are preferred. The sodium salt is very particularly preferred. The quantities utilized in the agents used according to the present invention are equal by preference to 0.05 to 10 wt %, based on the entire agent, particularly preferably 0.1 to 5, and in particular 0.1 to 3 wt %.

Lastly, the agents used according to the present invention can also contain plant extracts (L).

These extracts are usually produced by extraction of the entire plant. In individual cases, however, it may also be preferred to produce the extracts exclusively from blossoms and/or leaves of the plant.

Lastly, the agents used according to the present invention can also contain plant extracts (L).

These extracts are usually produced by extraction of the entire plant. In individual cases, however, it may also be preferred to produce the extracts exclusively from blossoms and/or leaves of the plant.

With regard to the plant extracts usable according to the present invention, reference is made in particular to the extracts that are listed in the table beginning on page 44 of the 3rd edition of the Guideline for declaring the contents of cosmetic agents [Leitfaden zur Inhaltsstoffdeklaration kosmetischer Mittel] published by the Association of the personal hygiene and washing agents industry [Industrieverband Körperpflege- and Waschmittel e.V. (IKW)], Frankfurt.

According to the present invention the extracts from green tea, oak bark, nettle, hamamelis, hops, henna, chamomile, burdock root, horsetail, hawthorn, linden blossoms, almond, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, ginseng, and ginger root are especially preferred.

Particularly preferred are the extracts from green tea, oak bark, nettle, hamamelis, hops, chamomile, burdock root, horsetail, linden blossoms, almond, aloe vera, coconut, mango, apricot, lemon, wheat, kiwi fruit, melon, orange, grapefruit, salvia, rosemary, birch, lady's-smock, wild thyme, yarrow, restharrow, meristem, ginseng, and ginger root.

The extracts from green tea, almond, aloe vera, coconut, mango, apricot, lemon, wheat, kiwi fruit, and melon are very particularly suitable for the use according to the present invention.

Water, alcohols, and mixtures thereof can be used as extraction agents for manufacturing the aforesaid plant extracts. Among the alcohols, lower alcohols such as ethanol and isopropanol, but in particular polyvalent alcohols such as ethylene glycol and propylene glycol, both as the only extraction agent and mixed with water, are preferred. Plant extracts based on water/propylene glycol at a ratio from 1:10 to 10:1 have proven particularly suitable.

According to the present invention the plant extracts can be used in both pure and diluted form. If they are used in diluted form, they usually contain approx. 2 to 80 wt % active substance, and contain as a solvent the extraction agent or extraction agent mixture used to obtain them.

It may furthermore be preferred to use mixtures of several, in particular two, different plant extracts in the agents used according to the present invention.

In addition, it may prove advantageous if penetration adjuvants and/or swelling agents (M) are contained in the agents used according to the present invention. To be included thereamong are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

A further subject of the present invention is a method for positively influencing the natural pigmentation process of skin and/or skin appendages, in particular for stimulating the natural pigmentation process, in particular the melanogenesis and/or pigmentation of hair, in order to prevent and/or decrease hair graying and/or to repigment grayed hair, wherein a combination of L-carnitine and/or an L-carnitine derivative with dihydroquercetin and/or a dihydroquercetin derivative is brought into contact topically with hair and/or skin.

With regard to further preferred embodiments of the method according to the present invention, the statements made about the uses according to the present invention apply mutatis mutandis.

### Example 1

### Demonstrating Differential Expression of Melanogenesis-Relevant Genes

Ligands participating in melanogenesis, such as SCF or alpha-MSH (melanocyte stimulating hormone alpha), bind to a variety of receptors by means of which the corresponding signal is delivered into the interior of the cell. The receptor for SCF is ckit, the receptor for alpha-MSH is MCR-1 (melanocortin receptor 1). Those substances that bring about a change in the expression of MCR-1 and/or ckit can influence melanogenesis. In the case of an induction (upregulation or stimulation) of the gene expression of the corresponding receptors, a stimulation of melanogenesis is to be assumed.

Gp100 is a protein that occurs in the membrane of melanosomes and stabilizes it. Because more melanin is produced in the cells after the application of substances that positively influence melanogenesis, an increase also occurs in the number of melanosomes required for transport. A substance that induces the gene expression of gp100 is therefore a pigmentation-stimulating active substance.

Particularly preferred substances that stimulate the natural pigmentation process of skin and/or skin appendages, in particular hair or hair follicles, are those both bring about the gene expression of MCR-1 and/or ckit, and induce the gene expression of gp100.

A determination of the extent of the change in gene expression after an application of such substances onto suitable cells/cell systems/tissue cultures can provide information regarding the efficacy of the active substance.

Differential gene expression was determined by means of quantitative RT-PCR. After three-dimensional organotypic hair follicle cell cultures from dermal papilla cells had been produced on microcarriers, the latter were incubated for 48 h with dihydroquercetin in two different concentrations. In order to carry out the PCR, firstly the RNA was isolated from the organotypic cell cultures using the RNeasy Mini Kit of the Qiagen company, and it was transcribed into cDNA by reverse transcription. In the context of the subsequent PCR reaction, which was carried out using gene-specific primers for the respective genes and which serves to amplify the desired gene segments, formation of the PCR products was detected online by way of a fluorescence signal, which is proportional to the quantity of PCR product formed. The stronger the expression of a specific gene, the greater the quantity of PCR product formed, and the higher the fluorescence signal.

The quantify the gene expression, the untreated control was set to equal 1, and expression of the genes to be determined was referred to it (X-times expression). Values that are greater than or equal to 1.8 times the expression, or less than or equal to 0.5 times the expression, of the untreated control are classified as being significantly differentially expressed. Values that are greater than or equal to 1.5 times the expression, or less than or equal to 0.7 times the expression, of the untreated control are classified as tending toward differential expression.

TABLE 1
Influence of dihydroquercetin on the expression of
melanogenesis-regulating genes

| | Conc. | ckit | | MCR1 | | gp100 | |
|---|---|---|---|---|---|---|---|
| | (μM) | M | SD | M | SD | M | SD |
| Untreated | | 1.00 | 0.19 | 1.00 | 0.34 | 1.00 | 0.53 |
| Dihydroquercetin | 10 | 1.32 | 0.39 | 2.56 | 0.44 | 0.89 | 0.33 |
| | 100 | 3.55 | 0.78 | 5.17 | 2.14 | 3.48 | 2.43 |

Especially at the 100 μM concentration of dihydroquercetin, the expression of all three genes was induced. The melanocortin 1 receptor was already significantly differentially expressed at a concentration of 10 μm dihydroquercetin.

### Example 2

### Stimulation of Melanin Synthesis

Melanin is a dye that is produced and stored in the melanosomes of melanocytes. Melanin gives hair its actual color, the color being produced by a mixture of two types of melanin, eumelanin and pheomelanin. Melanogenesis is a complicated synthesis process regulated in many ways. Firstly, tyrosine is converted by the enzyme tyrosinase into L-dihydroxyphenylalanine (L-DOPA), and then via multiple intermediate steps into the various melanin pigments. An active substance that positively influences melanogenesis and results in an elevated melanin content in the hair follicle melanocytes is particularly suitable for influencing the natural pigmentation process of skin and/or skin appendages, preventing hair graying, and/or stimulating pigmentation.

To investigate the melanin content, three-dimensional organotypic hair follicle cell cultures made up of dermal papilla cells, hair follicle melanocytes, and outer root sheath keratinocytes were treated for 7 days with dihydroquercetin (10 μM and 100 μM). Untreated cell cultures served as controls. After 4 and 7 days the hair follicle equivalents were homogenized and the melanin was extracted for 45 minutes using NaOH (1M)+10% DMSO at 100° C. Aliquots of the samples were then transferred into a 96-well plate, and extinction at 492 nm was measured. The increase in melanin content from day 0 to day 7 was evaluated by referring all the values to the untreated control at day 7 (=100%).

TABLE 2
Melanin content in hair follicle equivalents
after treatment with dihydroquercetin

| Melanin content (%) | |
|---|---|
| d 4 | d 7 |

| | Conc. (μM) | M | SD | M | SD |
|---|---|---|---|---|---|
| Untreated | | 100 | 28 | 181 | 86 |
| Dihydroquercetin | 10 μM | 100 | 28 | 254 | 58 |
| | 100 μM | 100 | 28 | 161 | 22 |

At both utilization concentrations, it was possible to increase melanin synthesis in the treated cultures considerably as compared with the control.

### Example 3

### Effect of Carnitine Tartrate

L-Carnitine and L-carnitine tartrate were investigated with regard to their effects on ATP synthesis the excretion of the growth factors HGF (hepatocyte growth factors) and KGF (keratinocyte growth factor). ATP (adenosine triphosphate) is the universal storage form for chemical energy in cells. Release of the phosphate groups yields ADP and Pi (inorganic phosphate). This reaction is highly exergonic, i.e. energy is released. ATP is produced in the cellular oxidative breakdown of fats, carbohydrates, and proteins. It serves as an energy supply for biochemical syntheses (including melanin synthesis), for transport operations (active transport), and for mechanical work. These processes are endergonic, i.e. they proceed only when energy is delivered. HGF and KGF are important growth factors with which the dermal papilla controls hair growth and the hair cycle, to which pigment production in the hair follicle is linked in particular fashion. Melanin formation takes place exclusively in the anagen phase of the hair cycle. In addition, a variety of publications have discussed the effect of HGF on DNA synthesis, on growth, and on the differentiation of melanocytes.

The ATP determination was performed using the ATPlite™-M assay (Packard). The test principle of this assay is based on the fact that the luciferase of Photinus pyralis catalyzes a reaction in which D-luciferin is converted into oxyluciferin in the presence of ATP. This reaction causes the emission of green light, which can be measured with a luminometer. The emitted bioluminescent light is proportional to the quantity of ATP present.

The ATP activity determination was performed in organotypic cell cultures made up of three-dimensionally cultivated dermal papilla cells. The treatment with carnitine and carnitine tartrate occurred over 24 hours, against an untreated control.

TABLE 3
ATP content in hair follicle cell cultures
after treatment with the amino acid mixture

ATP (%)

| | Conc (%) | M | SD |
|---|---|---|---|
| Untreated | | 100 | 4 |
| Carnitine | 0.05% | 212 | 51 |
| | 0.1% | 160 | 28 |
| Carnitine tartrate | 0.01% | 195 | 20 |
| | 0.05% | 102 | 28 |

At the utilization concentrations of 0.05% and 0.1% carnitine and 0.01% carnitine tartrate, it was possible to increase the ATP content in the treated cultures significantly as compared with the control.

The excretion of HGF and KGF can be quantified with the aid of a commercially obtainable ELISA kit. For this, organotypic hair follicle cell cultures made up of dermal papilla cells, hair follicle melanocytes, and outer root sheath keratinocytes are incubated for 72 hours with carnitine tartrate, and the concentration of HGF and KGF in the medium is determined.

TABLE 4
Relative excretion of HGF and KGF (%)

| | |
|---|---|
| HGF (%) | KGF (%) |

| | Conc. (%) | M | SD | M | SD |
|---|---|---|---|---|---|
| Untreated | | 100 | 6 | 100 | 13 |
| Carnitine | 0.01% | 105 | 2 | 120 | 11 |
| Carnitine tartrate | 0.005% | 141 | 46 | 123 | 9 |

At the selected utilization concentrations, content of growth factors in the treated cultures considerably raised as compared with the untreated control.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A method for positively influencing the natural pigmentation process of skin or appendages thereof, comprising:
topically contacting the skin or appendages thereof with a combination of carnitine and/or a derivative thereof with dihydroquercetin and/or a derivative thereof.

2. The method according to claim 1, wherein the step of topically contacting the skin or appendages thereof with the combination stimulates the natural pigmentation process of hair.

3. The method according to claim 1, wherein the step of topically contacting the skin or appendages thereof with the combination stimulates at least one sub-step of the natural pigmentation process.

4. The method according to claim 1, wherein the step of topically contacting the skin or appendages thereof with the combination stimulates the pigmentation of hair.

5. The method according to claim 1, wherein the step of topically contacting the skin or appendages thereof with the combination stimulates melanogenesis.

6. The method according to claim 1, wherein the step of topically contacting the skin or appendages thereof with the combination reduces hair graying.

7. The method according to claim 1, wherein the step of topically contacting the skin or appendages thereof with the combination repigments grayed hair.

8. The method according to claim 1, wherein the carnitine derivative is selected from carnitine tartrate, acetylcarnitine, carnitine fumarate, carnitine citrate, and lauroylcarnitine.

9. The method according to claim 1, wherein the ratio of the quantity of dihydroquercetin and/or a dihydroquercetin derivative to the total quantity of carnitine or of the carnitine derivative ranges between 10:1 and 1:10.

10. The method according to claim 1, wherein the dihydroquercetin and/or derivative thereof is part of a cosmetic agent that topically contacts the skin or appendages there of and contains the dihydroquercetin and/or derivative thereof in a total quantity of 0.000001 to 3 wt % based on the total weight of the agent.

11. The method according to claim 1, wherein carnitine and/or derivative thereof is part of a cosmetic agent that topically contacts the skin or appendages there of and contains carnitine and/or the carnitine derivative in a total quantity of 0.000001 to 10 wt % based on the total weight of the agent.

12. A hair treatment agent containing
a. 0.1 to 90 wt % of at least one monovalent alcohol from the group of ethanol, n-propanol, isopropanol, n-butanol;
b. 0 to 10 wt % of at least one gel former;
c. L-carnitine and/or derivative thereof; and
d. dihydroquercetin and/or derivative thereof.
